(19) United States
(12) Patent Application Publication
(10) Pub. No.: US 2011/0196341 A1
(43) Pub. Date: Aug. 11, 2011

(54) DEFLATION INDICATOR FOR A MEDICAL DEVICE BOLSTER

(75) Inventor: Glade H. Howell, Sandy, UT (US)

(73) Assignee: C. R. Bard, Inc., Murray Hill, NJ (US)

(21) Appl. No.: 13/024,046

(22) Filed: Feb. 9, 2011

Related U.S. Application Data
(60) Provisional application No. 61/302,620, filed on Feb. 9, 2010.

(51) Int. Cl. A61M 39/08; A61M 39/22

(52) U.S. Cl. 604/514; 604/100.01; 604/99.04

(57) ABSTRACT

A deflation indicator system for visually indicating an inflation status of a balloon-type or other inflatable internal bolster of a medical device is disclosed. The indicator system enables an observer to ascertain from an external vantage point whether the internal bolster of the indwelling device is inflated. In one embodiment, the medical device comprises an elongate body defining a lumen, and an inflatable internal bolster for securing a portion of the medical device within the patient. A deflation indicator is also disclosed, including a flexible membrane in fluid communication with the internal bolster. The membrane is included in an external portion of the medical device. The membrane assumes a first concavely-shaped position when the internal bolster is deflated. Correspondingly, the membrane assumes a second convexly-shaped position when the internal bolster is inflated. The positions of the membrane are externally observable to indicate the inflation status of the internal bolster.

### CROSS-REFERENCE TO RELATED APPLICATIONS

This application claims the benefit of U.S. Provisional Patent Application No. 61/302,620, filed Feb. 9, 2010, and entitled “Deflation Indicator for a Feeding Device Bolster,” which is incorporated herein by reference in its entirety.

### BRIEF SUMMARY

Briefly summarized, embodiments of the present invention are directed to a deflation indicator system for visually indicating an inflation status of a balloon-type or other inflatable internal bolster of a medical device is disclosed. The indicator system enables an observer to ascertain from an external vantage point whether the internal bolster of the indwelling device is inflated. This in turn helps prevent inadvertent removal of the device from within the patient due to unintended bolster deflation.

In one embodiment, the medical device comprises an elongate body defining a lumen extending from a proximal end to a distal end, and an inflatable internal bolster for securing a portion of the medical device within the patient. A deflation indicator is also disclosed, including a flexible membrane in fluid communication with the internal bolster. The membrane is included in an external portion of the medical device. The membrane assumes a first dome-shaped position when the internal bolster is deflated. Correspondingly, the membrane assumes a second, opposing dome-shaped position when the internal bolster is inflated. The positions of the membrane are externally observable to indicate the inflation status of the internal bolster.

These and other features of embodiments of the present invention will become more fully apparent from the following description and appended claims, or may be learned by the practice of embodiments of the invention as set forth hereinafter.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A is a cross sectional side view of a feeding device including a deflation indicator system in a first configuration, according to one embodiment;

FIG. 1B is a side view of a proximal portion of the feeding device of FIG. 1A, showing the deflation indicator system in the first configuration;

FIG. 2A is a cross sectional side view of the feeding device of FIG. 1A with the deflation indicator system in a second configuration, according to one embodiment;

FIG. 2B is a side view of a proximal portion of the feeding device of FIG. 2A, showing the deflation indicator system in a second configuration;

FIG. 3 is a partial cross sectional side view of a feeding device including a deflation indicator system according to one embodiment;

FIG. 4A is a top view of the feeding device of FIG. 3, showing the deflation indicator system in a first configuration;

FIG. 4B is a top view of the feeding device of FIG. 3, showing the deflation indicator system in a second configuration;

FIG. 5A is a partial cross sectional side view of a feeding device including a deflation indicator system in a first configuration according to one embodiment;

FIG. 5B is a partial cross sectional side view of the feeding device of FIG. 5A, showing the deflation indicator system in a second configuration;

FIG. 6 is a partial cross sectional side view of a feeding device including a deflation indicator system according to one embodiment;

FIG. 7A is a partial cross sectional side view of a feeding device including a deflation indicator system in a first configuration according to one embodiment;

FIG. 7B is a partial cross sectional side view of the feeding device of FIG. 7A including the deflation indicator system in a second configuration;

FIG. 8 is a partial cross sectional side view of a feeding device including a deflation indicator system according to one embodiment;

FIG. 9A is a perspective view of a portion of a feeding device including a deflation indicator system in one configuration according to one embodiment; and

FIG. 9B is a perspective view of the portion of the feeding device of FIG. 9A including the deflation indicator system in another configuration.

### DETAILED DESCRIPTION OF SELECTED EMBODIMENTS

Reference will now be made to figures wherein like structures will be provided with like reference designations. It is understood that the drawings are diagrammatic and schematic representations of exemplary embodiments of the present invention, and are neither limiting nor necessarily drawn to scale.

For clarity it is to be understood that the word “proximal” refers to a direction relatively closer to a clinician using the device to be described herein, while the word “distal” refers to a direction relatively further from the clinician. For example, the end of a feeding device placed within the body of a patient is considered a distal end of the device, while the device end remaining outside the body is a proximal end of the feeding device. Also, the words “including,” “has,” and “having,” as used herein, including the claims, shall have the same meaning as the word “comprising.”

Embodiments of the present invention are generally directed to a medical device, such as a feeding device, including an indicator system for visually indicating an inflation/deflation status of a balloon-type or other inflatable internal bolster of the device. The indicator system enables an observer to ascertain from an external vantage point whether the inflatable bolster of the indwelling device is sufficiently inflated and is therefore able to prevent inadvertent removal of the device from within the patient due to unintended bolster deflation.

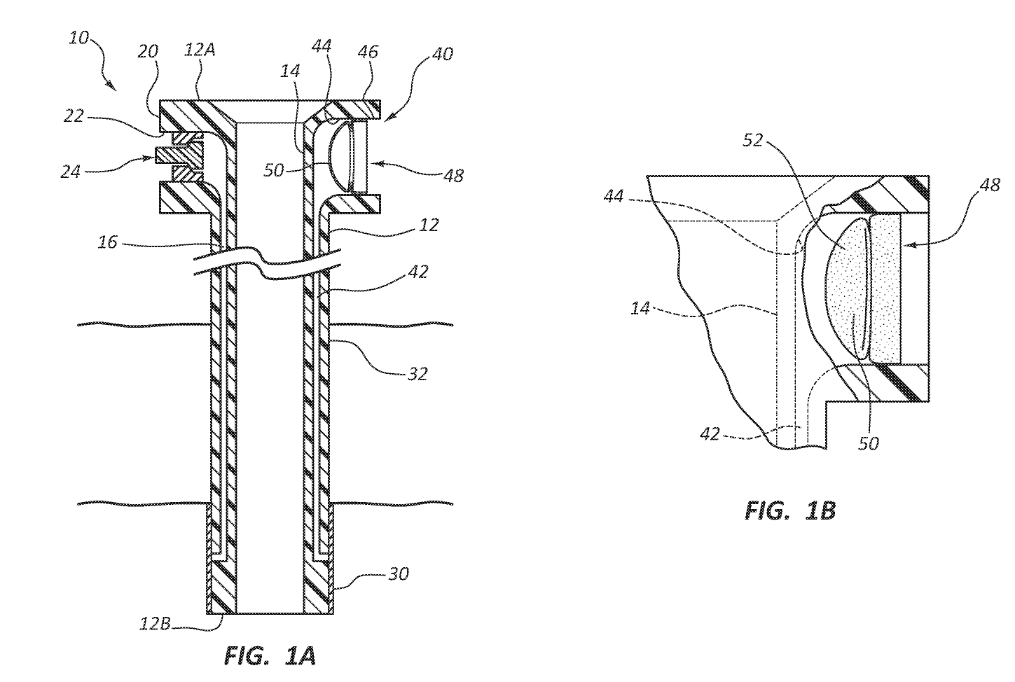

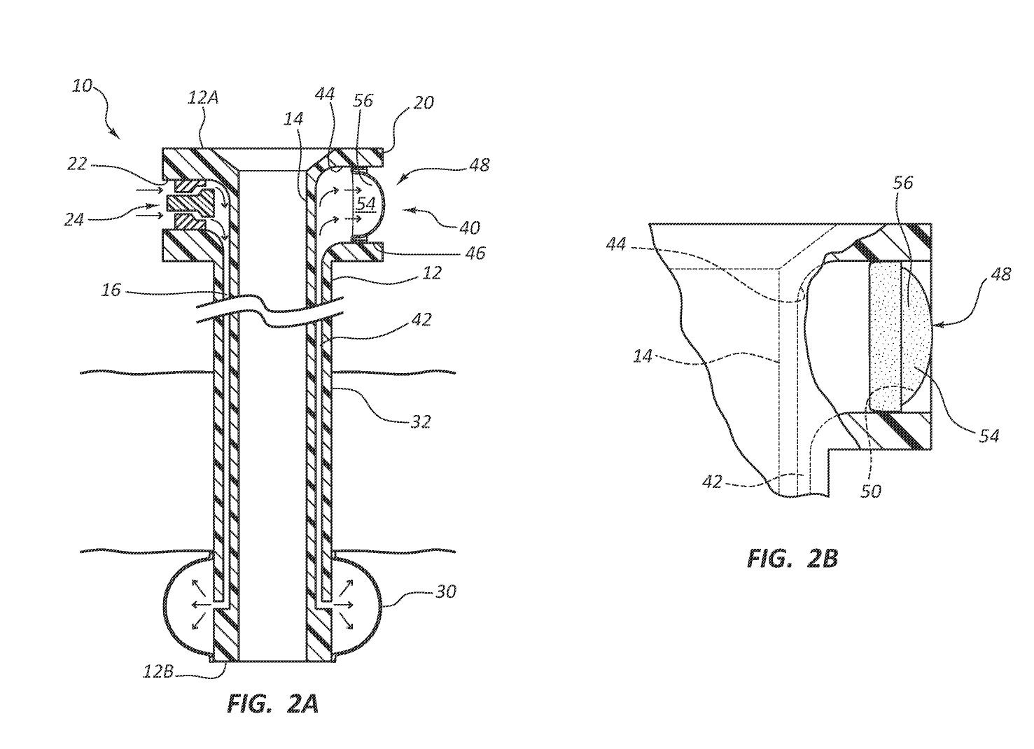

FIGS. 1A-2B depict one embodiment of a gastrostomy device, or feeding device, generally indicated at 10. In particular, the device 10 includes an elongate body 12 defining a proximal end 12A, a distal end 12B, and a feeding lumen 14 extending between the proximal and distal ends. An inflation lumen 16 is also defined by the device body 12 and extends longitudinally adjacent the feeding lumen 14. Note that the inflation lumen 16 can be configured in a number of ways, including as a lumen offset with respect to the feeding lumen, concentric therewith, or interior thereto. The feeding lumen can also be configured in various ways in addition to what is shown and described herein. More generally, note that the feeding device is but one example of an indwelling medical device employing an inflatable bolster to secure the device within the body of the patient; other devices are therefore contemplated as benefitting from the principles described herein.

A proximal head portion 20 is included proximate the proximal end 12A of the feeding device body 12. The head portion 20, which remains external to the patient after placement of the feeding device 10, provides an inlet for the feeding lumen 14 through which nutritional solution or other substance can be passed into the stomach or other suitable internal portion of the patient. As shown in FIG. 1A, in the present embodiment the feeding device 10 is inserted into the patient via a stoma 32 so as to provide access to the stomach of the patient.

In the illustrated embodiment, the head portion 20 includes an inflation port 22 in fluid communication with the inflation lumen 16. A valve 24 is included in the inflation port 22. An inflatable internal bolster is included proximate the distal end 12B of the feeding device body 12. In the present embodiment, the internal bolster is implemented as an annular balloon 30, which is in fluid communication with the inflation lumen 16. The balloon 30 is shown in a deflated state in FIG. 1A to enable insertion of the feeding device 10 into a patient via the stoma 32. The balloon 30 is shown in an inflated state in FIG. 2A after the feeding device 10 has been inserted into the patient such that the balloon acts as an internal bolster to prevent inadvertent removal of the feeding device from the patient. The balloon 30 can be inflated by injecting air, a liquid such as saline, or other suitable inflation component through the inflation port 22 and inflation lumen 16. The valve 24 is positioned in the inflation port 22 and configured to enable selective inflation of the balloon 30 and prevent unintended fluid escape through the inflation port 22 once the balloon is inflated. The inflation port and valve can be configured in one of any number of ways.

According to the present embodiment, the feeding device 10 further includes a deflation indicator system, generally indicated at 40, for visually indicating the inflation status of the balloon 30. Ascertaining the inflation status of the balloon 30 is useful for preventing inadvertent removal of the feeding device 10 when the balloon has unknowingly deflated and is unable to maintain the device in position within the patient.

In the illustrated embodiment, the deflation indicator system 40 includes a connecting lumen 42 defined longitudinally adjacent the feeding lumen 14, as shown in FIGS. 1 and 2. Though shown as separately formed here, in one embodiment the connecting lumen 42 and the inflation lumen 16 are integrally formed with one another, such as in the case when both are defined by a concentric lumen circumferentially defined about the feeding lumen 14, for instance. In either case above, the inflation lumen and connecting lumen are in at least indirect fluid communication with one another via the balloon 30. Other possible configurations for these lumens are possible.

The connecting lumen 42 is in fluid communication with the balloon 30 on a distal end thereof and with a cavity 44 on a proximal end thereof. The cavity 44 is included in the head portion 20 of the feeding device 10 and defines an outer perimeter 46. An indicator 48, which in the present embodiment includes a reversible non-permeable, dome-shaped membrane, is sealably attached to the outer perimeter 46 of the cavity such that fluid passage therethrough or therearound is prevented. The proximal head portion 20 in the present embodiment includes a translucent material so as to enable visual observation of the indicator 48 disposed in the cavity 44.

The indicator 48 is bi-positional in the present embodiment and is biased inward to a first position, which corresponds to the internal bolster balloon 30 being in a deflated state, as shown in FIG. 1A. In this first position, a first surface 50 of the indicator 48 is visible, as best seen in FIG. 1B. In particular, the indicator first surface 50 includes a first indicium 52 thereon, which in the present embodiment includes coloring of the first surface. In one embodiment, the color of the first indicium 52 is red, but any color may be used. Also, the first indicium 52 in other embodiments can include other designs, patterns, symbols, letters, numbers, etc. In one embodiment, the shape, position, or other configuration of the indicator itself may serve as a sufficient indicium.

The indicator 48 is configured such that it is pushed and folded outward to a second position via fluid pressure acting thereon (via the connecting lumen 42) when the internal bolster balloon 30 is inflated, as is shown in FIG. 2A. Movement of the indicator 48 to the second position causes a second surface 54 of the indicator 48 to be visible through the cavity 44, as shown in FIG. 2B. The second surface 54 includes a second indicium 56, which in the present embodiment is the color white, but which may be any other color, design, pattern, symbol, letter, number, etc. that is differentiated from the first indicium 52 disposed on the first surface 50 of the indicator 48. Thus, the indicator 48 can be thought of as assuming a substantially hemispherical, cross sectionally concave shape when in the first position, as seen in FIG. 1A, and a substantially hemispherical, cross sectionally convex shape in the second position, as seen in FIG. 2A. Note that the substantially fluid-impermeable membrane of the indicator 48 can include various suitable materials, including polyisoprene, silicone, etc.

In view of the foregoing, the deflation indicator system 40 as described herein can be employed to determine the inflation status of the balloon 30 when the feeding device 10 is disposed within a patient as shown in FIGS. 1A and 2A. In particular, if the balloon 30 is inflated so as to serve as the internal bolster of the in-place feeding device 10, then the fluid pressure from the inflated balloon is also present at the cavity 44 and indicator 48 via the connecting lumen 42. This pressure causes the indicator to be pushed outward to the second position (FIGS. 2A, 2B) to show the second indicium 56 located on the second surface 54. Visual observation of the second indicium 56 of the indicator 48 thus indicates to an observer that the balloon 30 is sufficiently inflated as an internal bolster to prevent the feeding device 10 from being inadvertently removed.

If the indicator 48 is observed as being in the first position shown in FIGS. 1A and 1B, however, there is insufficient fluid pressure present at the cavity 44 and indicator, which equates to a deflated state of the balloon 30. A clinician observing the first indicium 52 visible when the indicator 48 is in the first position can thus conclude that the balloon 30 is deflated and take corrective steps as needed.

Note that the indicator can be configured so as to change from its first position to its second position according to a predetermined fluid pressure acting on the indicator, and that the predetermined fluid pressure can vary according to application and/or feeding device design, or other considerations as appreciated by one skilled in the art. Note also that the deflation indicator system can be included with feeding and other devices that employ balloons of many different configurations in addition to what is shown and described herein.

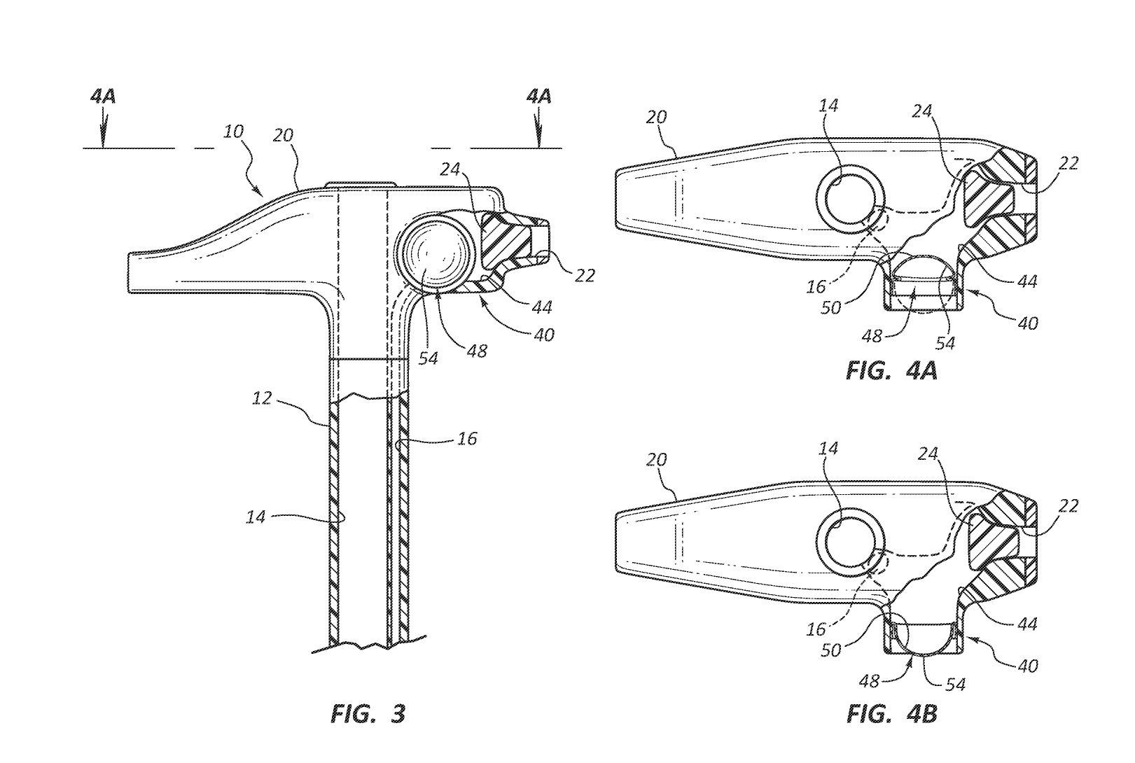

FIGS. 3-4B show various details of the deflation indicator system 40 according to another embodiment, wherein the indicator system and the inflation port are in fluid communication with the balloon 30 via a single lumen, i.e., the inflation lumen 16. As such, no connecting lumen for exclusive use by the deflation indicator system 40 is needed, as the inflation lumen 16 provides not only a conduit for filling the balloon 30 via the inflation port 22 and valve 24 but a lumen for the transfer of fluid pressure to the indicator 48. As seen, the cavity 44 is sized so as to provide space for the valve 24 and the indicator 48. Note that the valve 24 can include a spring or other device for maintaining the valve in a seated position against surfaces of the inflation port 22.

As before, the indicator 48 is bi-positional in one of a first position (FIG. 4A) to indicate deflation of the internal bolster balloon 30, and a second position (FIG. 4B) to indicate inflation thereof. It is noted that, as a flexible, substantially impermeable membrane in one embodiment, the indicator 48 can assume a position in between the first and second positions should fluid pressure circumstances allow. Such a position, for instance, could indicate that the internal bolster is not fully deflated, but needs to be further inflated to full capacity.

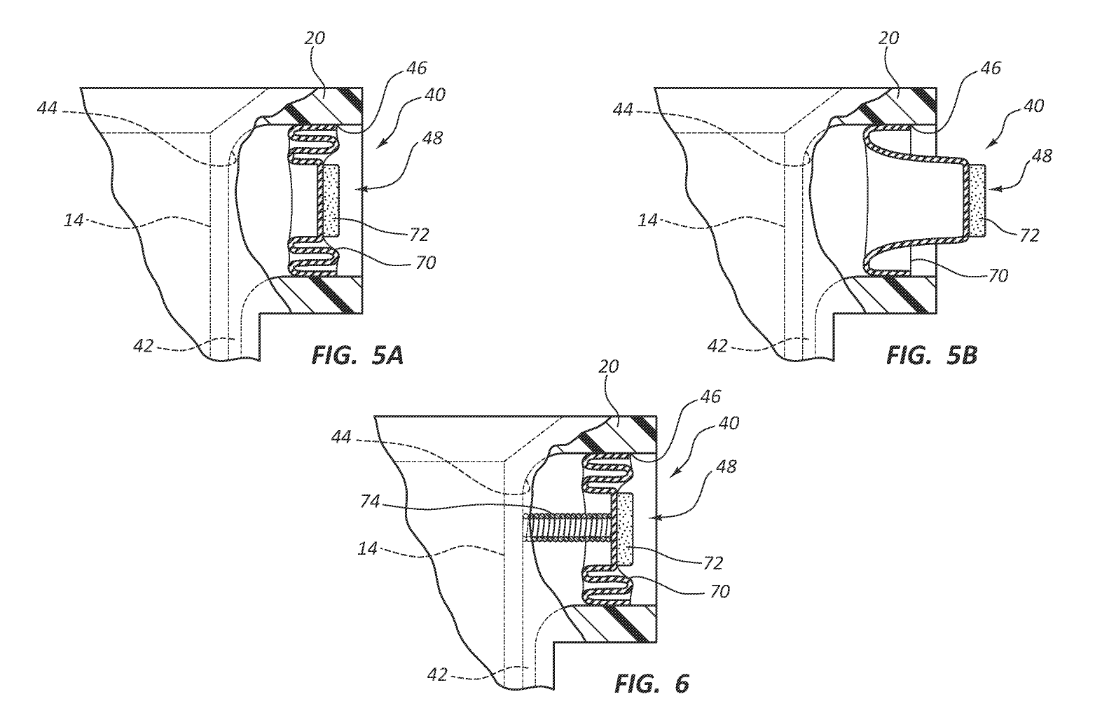

FIGS. 5A and 5B depict the deflation indictor system 40 according to another embodiment, wherein the indicator 48 includes a rippled membrane 70 to allow for relatively greater extension when indicating an inflated state of the internal bolster, such as in FIG. 5B. A colored cap serves as an indicium 72 that is visible when the membrane 70 is extended to indicate inflation of the internal bolster balloon. FIG. 6 shows a similar embodiment to that of FIGS. 5A and 5B, wherein a spring 74 is operably attached to the membrane 70 to assist with retraction thereof in the event of balloon deflation.

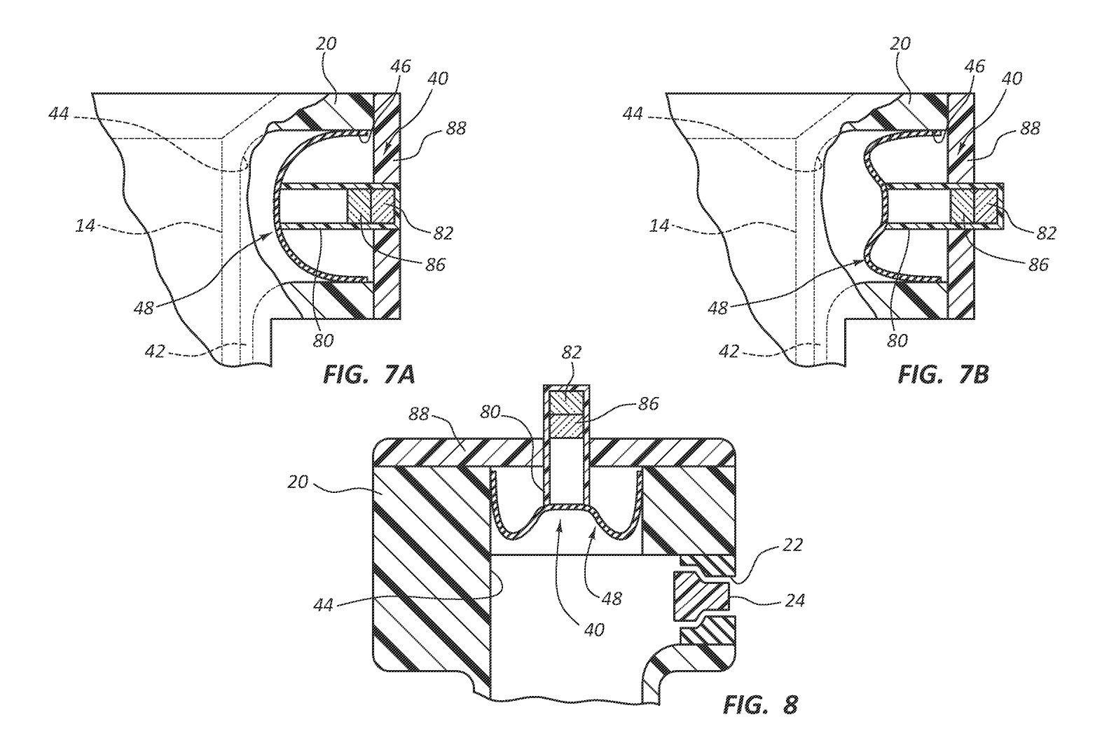

FIGS. 7A and 7B depict the deflation indictor system 40 according to another embodiment, wherein a rod 80 is operably attached to the membrane indicator 48. As such, the rod 80 in the illustrated embodiment can reside within the head portion 20 of the feeding device when the internal bolster balloon is deflated so as not to be visible to an observer. Correspondingly, the rod 80 can extend from the head portion 20, through a cover 88 for instance, when the balloon is inflated such that an indicium 82 is visible, as shown in FIG. 7B. The rod 80 can further include another indicium 86 that is visible when the internal bolster balloon is over-inflated, as seen in FIG. 8. This will indicate to the observer that the balloon should be deflated until proper inflation is achieved. For instance, the indicium 82 in one embodiment can include a green band, while the indicium 86 includes a red band. FIG. 8 further shows that the deflation indicator system 40 can be disposed in other locations and configurations on the feeding device, such as the vertical placement shown here.

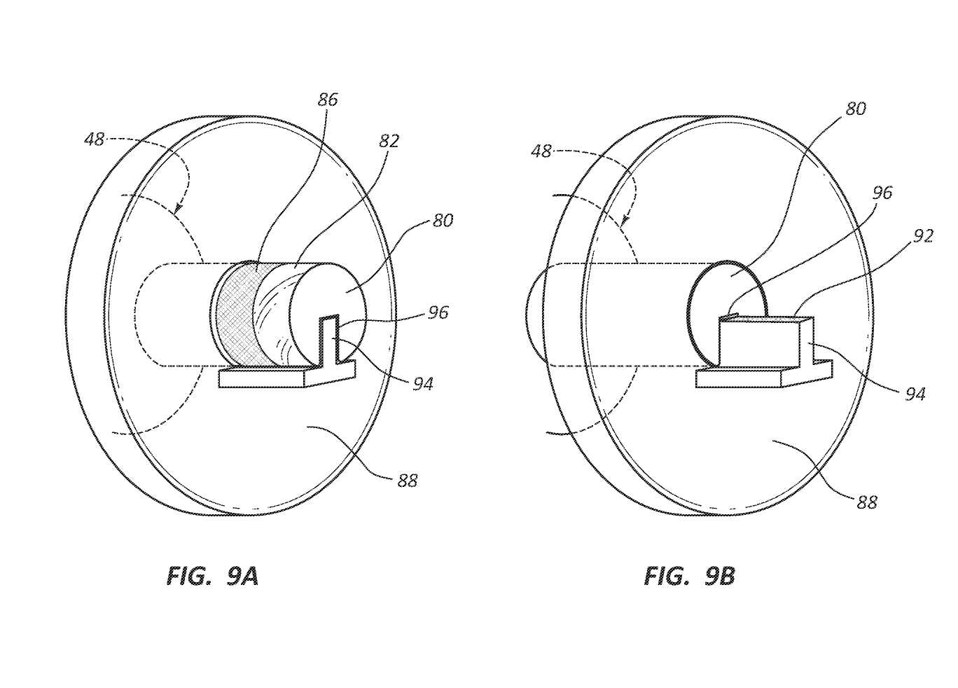

FIGS. 9A and 9B depict the deflation indictor system 40 according to another embodiment, wherein the rod 80 includes a groove 96 to enable the rod to slide along a rail 94 attached to the cover 88 or other portion of the feeding device. In FIG. 9A, the rod 80 is fully extended along the rail 94 such that the indicium 82 indicating balloon inflation and indicium 86 indicating over-inflation are visible. In FIG. 9B, the rod 80 is retracted, indicating deflation of the balloon. An indicium 92 disposed on the rail 94 is visible in this case, and visually indicates balloon deflation.

Note that the particular configuration of the rod and indicia described above can be varied from what is shown and described. Indeed, the size, shape, position, etc. of the indicators and indicia can take various forms to shows balloon inflation status.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. A medical device, comprising:
an elongate body defining a lumen extending from a proximal end to a distal end;
an inflatable internal bolster for securing a portion of the medical device within the patient; and
a deflation indicator including:
a flexible membrane in fluid communication with the internal bolster and disposed on an external portion of the medical device, wherein the membrane assumes a first concavely-shaped position when the internal bolster is deflated, and wherein the membrane assumes a second convexly-shaped position when the internal bolster is inflated, the position of the membrane being externally observable to indicate an inflation status of the internal bolster.

2. The medical device as defined in claim 1, wherein the deflation indicator is included in an external head portion of the medical device, at least a portion of the head portion being translucent so as to enable observation of the membrane.

3. The medical device as defined in claim 1, wherein the indicator is housed in a cavity of the external head portion, the cavity defining a perimeter, the indicator sealed about the perimeter.

4. The medical device as defined in claim 1, wherein at least a portion of the membrane defines a substantially domed shape when in at least one of the first and the second positions.

5. The medical device as defined in claim 1, wherein a first surface of the membrane is visible when the membrane is in the first position, the first surface including a first indicium thereon.

6. The medical device as defined in claim 5, wherein a second surface of the membrane is visible when the membrane is in the second position.

7. The medical device as defined in claim 6, wherein the second surface of the membrane includes a second indicium visible when the second surface is visible, and wherein at least one of the first and second indicia includes a colored surface.

8. The medical device as defined in claim 1, wherein the medical device includes a gastrostomy device, and wherein the membrane is in fluid communication with the internal bolster via a connecting lumen.

9. A method for inserting a medical device into a patient, the method comprising:
providing the medical device disclosed in claim 1;
inserting a distal portion of the medical device into the patient such that the internal bolster is disposed within the patient; and
inflating the internal bolster to secure the medical device within the patient and such that the membrane of the deflation indicator is disposed in the second position,
wherein the membrane is externally observable such that deflation of the internal bolster is indicated when the membrane is disposed in the first position.

10. The method for inserting as defined in claim 9, further comprising:
attending to the medical device to re-inflate the internal bolster to dispose the membrane in the second position.

11. The method for inserting as defined in claim 9, wherein a first indicium of the membrane is visible when the membrane is disposed in the first position.

12. The method for inserting as defined in claim 11, wherein inflating the internal bolster further comprises:
inflating the internal bolster to secure the medical device within the patient and such that a second indicium of the membrane is visible when the membrane is disposed in the second position.

13. A deflation indicator for a medical device, the medical device including an inflatable internal bolster for securing a portion of the medical device within the patient, the deflation indicator comprising:
a bi-positional membrane in fluid communication with the internal bolster and included with an external portion of the medical device, wherein the membrane assumes a substantially dome-shaped first position when the internal bolster is deflated, and wherein the membrane assumes an opposing substantially dome-shaped second position when the internal bolster is inflated.

14. The deflation indicator as defined in claim 13, wherein a perimeter of the membrane is attached within a cavity of a head portion of the medical device, and wherein the membrane is visually observable within the cavity.

15. The deflation indicator as defined in claim 14, wherein the head portion is translucent to enable external observation of the membrane, and wherein the membrane assumes a cross sectionally concave shaped when in the first position and a cross sectionally convex shape when in the second position.

16. The deflation indicator as defined in claim 13, wherein the membrane includes a first indicium visible only when the membrane assumes the first position, the first indicium including a red color portion of the membrane.

17. The deflation indicator as defined in claim 16, wherein the membrane includes a second indicium visible when the membrane assumes the second position.

18. A feeding device, comprising:
an elongate body defining a feeding lumen that extends from a proximal end to a distal end of the body, the body extendable through a stoma defined in a body of a patient;
an inflatable balloon for providing an internal bolster for the feeding device; and
a deflation indicator system including:
a connecting lumen in fluid communication with the balloon; and
a bi-positional dome-shaped indicator in fluid communication with the connecting lumen and including a first indicium that is only visible when the indicator is in a first position, the first position corresponding to a deflated state of the balloon and a second indicium that is visible when the indicator is in a second position, the second position corresponding to an inflated state of the balloon.

19. The feeding device as defined in claim 18, wherein the indicator is included in a cavity disposed in a proximal head portion of the feeding device body, and wherein the membrane is in fluid communication with the internal bolster via a connecting lumen.

20. The feeding device as defined in claim 19, further comprising an inflation port including a valve.

21. The feeding device as defined in claim 20, wherein the inflation port and the indicator are in fluid communication with the internal bolster via the connecting lumen.

22. A deflation indicator for a medical device, the medical device including an inflatable internal bolster for securing a portion of the medical device within the patient, the deflation indicator comprising:
a rippled membrane in fluid communication with the internal bolster and included with an external portion of the medical device, wherein the membrane assumes a substantially collapsed first position when the internal bolster is deflated, and wherein the membrane assumes an extended second position when the internal bolster is inflated.

23. The deflation indicator as defined in claim 22, wherein the membrane is substantially fluid-impermeable and wherein an indicium is included with the membrane that is visible when the membrane assumes the second position.

24. A deflation indicator for a medical device, the medical device including an inflatable internal bolster for securing a portion of the medical device within the patient, the deflation indicator comprising:
a substantially fluid-impermeable membrane in fluid communication with the internal bolster and disposed in an external portion of the medical device, wherein the membrane is capable of moving from a first position when the internal bolster is deflated to at least a second position when the internal bolster is inflated, the membrane being biased to return to the first position when the internal bolster is deflated; and
a rod movable with the membrane such that the rod assumes a first rod position when the membrane is in the first position and such that the rod assumes a second rod position when the membrane is in the second position.

25. The deflation indicator as defined in claim 24, wherein the rod includes at least a first colored band that is visible when the rod is in the second rod position to indicate an inflation status of the internal bolster.

26. The deflation indicator as defined in claim 24, wherein the membrane is capable of moving to a third position when the internal bolster is over-inflated, and wherein the rod assumes a third rod position when the membrane is in the third position.

27. The deflation indicator as defined in claim 24, wherein the rod includes a groove that receives a rail to enable the rod to slide along the rail when the rod moves from the first rod position to at least the second rod position.
